(19) United States
(12) Patent Application Publication
(10) Pub. No.: US 2005/0038367 A1
(43) Pub. Date: Feb. 17, 2005

(54) Adjustable knee stabilizer

(76) Inventors: Scott D. McCormick, Cincinnati, OH (US); Sherry A. Hinds, Goshen, OH (US)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/639,832

(22) Filed: Aug. 13, 2003

(51) Int. Cl. A61F 5/00

(52) U.S. Cl. 602/26

(57) ABSTRACT

An adjustable knee stabilizer is provided having a body and a pair of integrated upper and lower straps, and a middle strap that is affixed to the body and extending away from the body and integrated straps. A pair of flexible stays are positioned about a patellar opening, and a pair of openings are defined in the body for collapsing during normal flexion of the knee.

### BACKGROUND OF THE INVENTION

The present invention relates to knee supports and, more specifically, to an adjustable knee support or stabilizer adapted for preventing injuries to the knee and/or supporting a knee that has been injured without unduly restricting circulation to the knee area or movement thereof.

It is well known that knee injuries are quite common among athletes and, perhaps not as frequently, among the general population as well. The knee area supports a wearer's body during walking, running, climbing, etc., and often becomes sore or damaged through overuse or an abnormal movement. Because it is exceptionally difficult to completely rest an injured knee, most people continue to walk, travel, and exercise on the injured knee, which typically extends the recovery time of the knee. In some cases, the knee requires additional support for an extended period of time for support and healing, sometimes permanently, depending on the activity. As such, a variety of knee braces and supports have been developed over the years.

A common knee support employs plastic or metal hinges on the lateral and medial sides of the knee. These supports are quite large and costly, and are typically reserved for extreme injuries that require exceptional support. Other supports employ a resilient sleeve, such as a sleeve made from neoprene or the like, that slips over the knee and provides compressive forces to the knee area. However, these sleeves do not provide adjustability and can be quite restrictive to circulation and movement of the knee.

Other braces and supports have been developed that can be wrapped about a wearer's knee and incorporate one or more straps that help secure the brace thereto. For example, U.S. Pat. No. 3,804,084 to Lehman discloses a knee support that includes a panel of padded felt material having top and bottom edges that are sized to extend around the thigh and calf, respectively. Openings are defined near the side edges that come together to form an opening for registration with the kneecap. A central overlock strap is provided that stretches over the kneecap in use to provide compressive force to the knee.

Another knee brace is shown in U.S. Pat. No. 5,759,167 to Shields, Jr. et al., which discloses a support wrap that includes a body and a pair of legs that extend laterally from the body. Connector tabs project from the legs and carry hook material so as to be connectable to loop material on the body portion.

Another knee support is shown in U.S. Pat. No. 5,865,777 to Detty, which discloses a knee support having a central portion and three integral mounting tabs. An opening is defined in the central portion for receiving the kneecap. Two cover strips hold respective stays and are configured to extend across the front of the knee and pass through a corresponding buckle. One disadvantage of the Detty support and similar braces is that the straps are integral to the body of the brace. This creates manufacturing deficiencies because the unique shape of the braces creates excessive waste during the cutting step of the manufacturing process. Another problem with braces having integral straps is that the straps are of the same material as the body of the brace and thus have the same functional properties, which may not be desirable for the straps, particularly considering the different functions that the straps play compared to the body of the brace.

Accordingly, while these knee braces provide satisfactory support, improvements can be made. In particular, the number, location, and material of the straps in the prior art leave room for significant advancements in terms of adjustability and manufacturing of the brace. In addition, advancements can be made for providing additional comfort and stability of the knee.

### BRIEF SUMMARY OF THE INVENTION

The adjustable knee stabilizer of the present invention balances the need for support of the knee area with the adjustability and comfort that is desirable and advantageous in order for a person to wear the stabilizer. The adjustable knee stabilizer of the present invention is easy to apply about the knee area, can be manufactured with improved efficiency of materials and less cost, and provides excellent support for the knee area without causing discomfort.

More specifically, an adjustable knee stabilizer according to one embodiment of the present invention comprises a body having a center portion, opposing lateral ends, and opposing top and bottom edges. The center portion defines a patellar opening for registration with the patella or kneecap. The body also defines a pair of collapsible openings proximate one of the lateral ends. In one embodiment, the openings have an elliptical shape and are positioned at a predetermined angle such that they collapse under knee flexion. The outer surface of the body is formed of a loop material, such as a VELCRO®-type loop material.

The knee stabilizer also includes a pair of integrated straps that are spaced apart and extend from one of the lateral ends of the body to preferably resemble a “U” shape. The straps include an upper strap adapted for wrapping about a wearer's leg above the patellar opening, and a lower strap adapted for wrapping about a wearer's leg below the patellar opening. Each strap has a hook material tab, such as VELCRO®-type hook material, for releasably attaching the straps to the body of the stabilizer by pulling the upper and lower straps around the knee stabilizer and the wearer's knee in use and adjustably securing the tabs to the outer surface of the body. The opposite outer side of the hook material tabs may include a retroflective material that improves night visibility and safety of the knee stabilizer.

The knee stabilizer also includes a center strap that is affixed to the body and extending from the opposite side of the body as the upper and lower straps. Advantageously, the center strap is formed from an elastic material that is preferably distinct or different than the body material and affixed to the body after the body has been formed. This process creates manufacturing benefits that are discussed more fully herein. According to one embodiment, the middle strap includes a hook material tab at its end, and is adapted for stretchably extending or wrapping behind a wearer's knee in use. The hook material tab of the center strap is releasably attached to the loop material outer surface of the body. As such, the patellar opening is not covered by the middle strap, and the center strap provides extra and balanced support for the knee.

The knee stabilizer of the present invention also includes a plurality of flexible stays adjacent the patellar opening on the lateral and medial sides of the body. The stays provide resistance to excessive knee flexion, and in one embodiment comprise a pair of stays on each side of the patellar opening. The stays may be straight or have a slightly curved shape or arc that is biased towards the patellar opening, which therefore allows a slight bend in the knee without excessive resistance. The stays are resiliently flexible, and are preferably formed of either an overlapping metal coil or an elongate polymeric member. In one embodiment, the stays are covered or encapsulated by stay covers that are attached to the outer surface of the body. The stay covers may be divided into sections in order to accommodate and separate more than one stay per cover.

Methods of manufacturing a knee stabilizer are also provided. The methods further distinguish the present invention and demonstrate the manufacturing efficiencies presented by forming a knee stabilizer according to the present invention.

Accordingly, the adjustable knee stabilizer of the present invention provides a protective support means for a wearer's knee that balances the need for support of the knee area with ease of manufacturing and adjustability that is desirable and advantageous in order for a person to wear the stabilizer with comfort.

### BRIEF DESCRIPTION OF THE SEVERAL VIEWS OF THE DRAWING(S)

Having thus described the invention in general terrns, reference will now be made to the accompanying drawings, which are not necessarily drawn to scale, and wherein:

FIG. 1 is a perspective view of an adjustable knee stabilizer in an in-use configuration according to one embodiment of the present invention;

FIG. 2 is a front plan view of the adjustable knee stabilizer of FIG. 1 in a laid-out configuration;

FIG. 3 is a side elevation view of the adjustable knee stabilizer of FIG. 1; and

FIG. 4 is a rear plan view of the adjustable knee stabilizer of FIG. 1.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

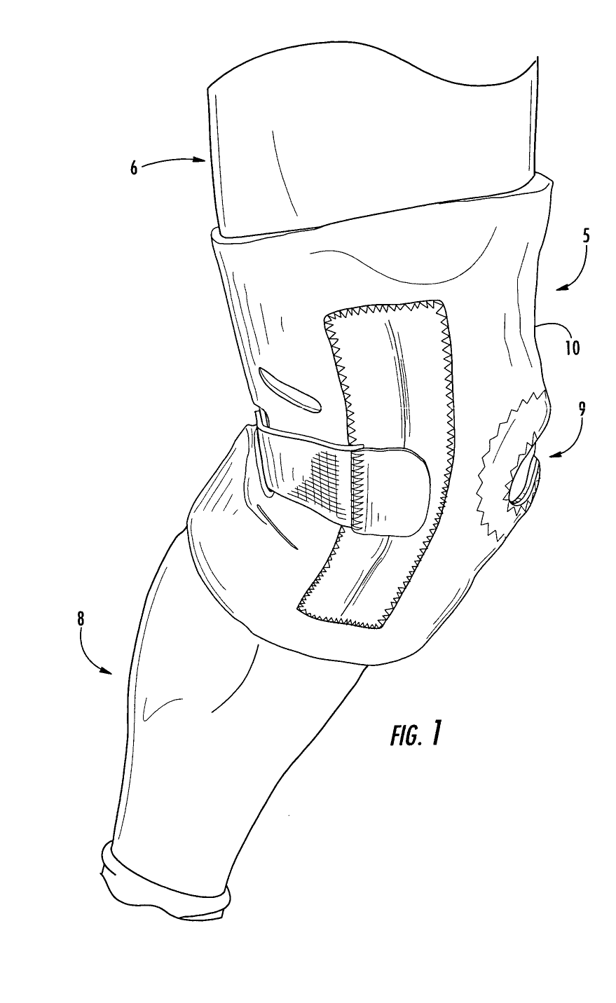

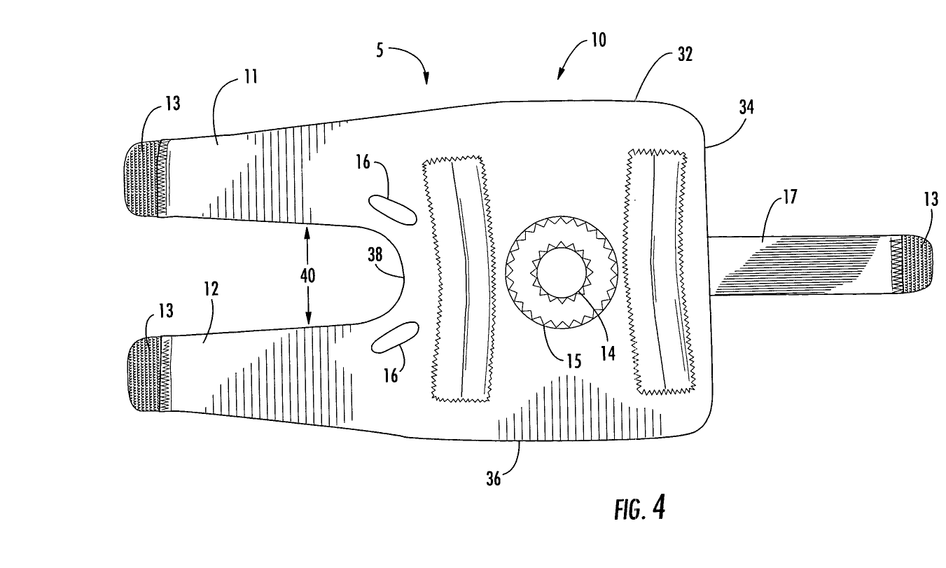

Turning now to the figures, FIGS. 1, 2, and 4 illustrate an adjustable knee stabilizer 5 for supporting a wearer's knee during exercise or normal activity. The knee stabilizer 5 is designed to wrap around a wearer's knee 9 between the wearer's thigh 6 and calf 8. The knee stabilizer 5 is adjustable through a plurality of straps that allow the knee stabilizer to be custom fitted depending on the size of the wearer's leg. In addition, the knee stabilizer 5 is easy to fit and remove, as the knee stabilizer is a generally planar structure that is wrapped around the wearer's leg instead of being pulled up the wearer's leg like a sleeve.

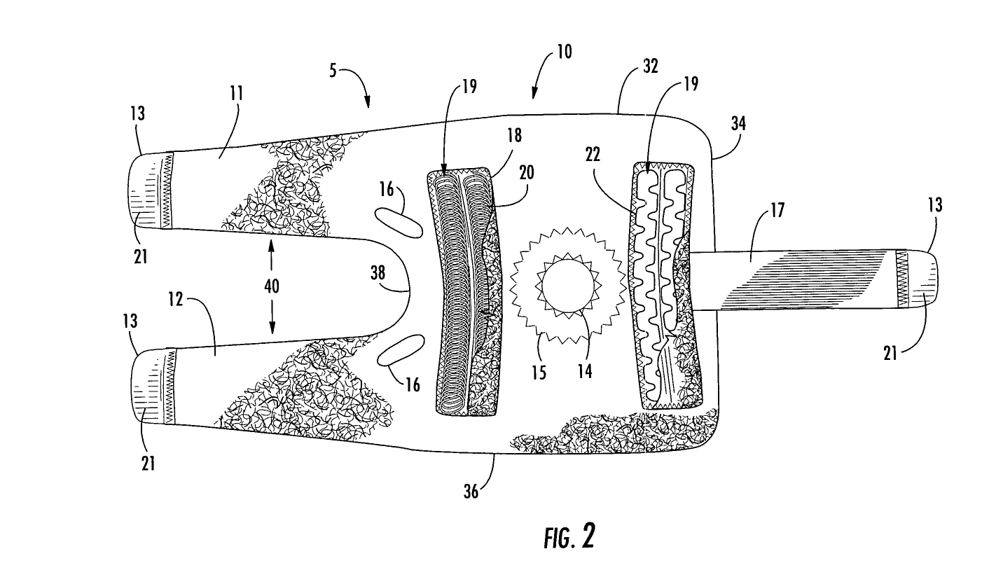

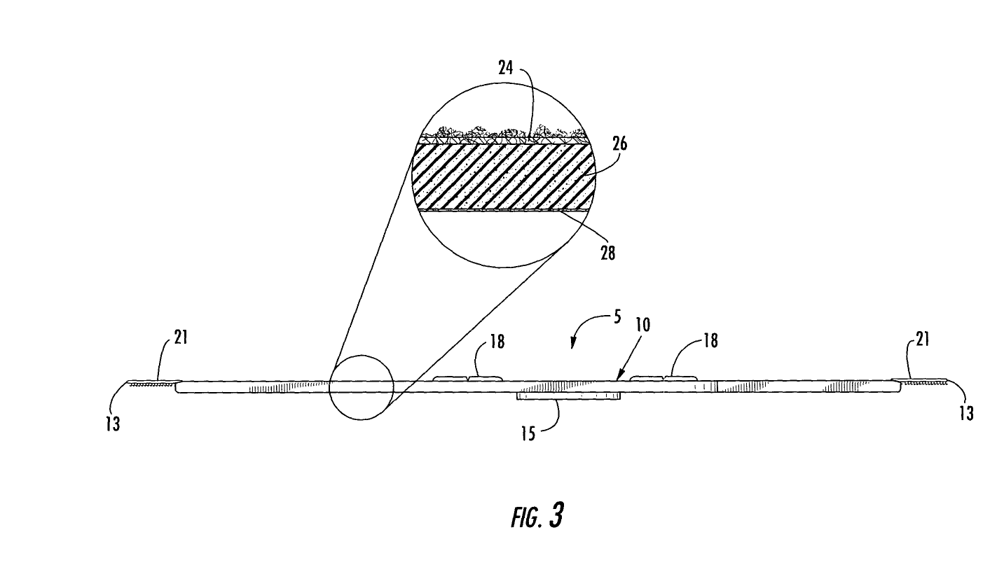

As shown in FIGS. 2 and 3, the knee stabilizer 5 according to one embodiment includes a body 10 constructed of a laminate material having multiple plies, including a soft inner layer 28, a neoprene middle layer 26, and a loop material outer layer 24. Advantageously, the inner layer is worn against the skin and has a low skin irritant, soft feel, and can have moisture-wicking properties, while the outer layer allows attachment of hook type fasteners, as discussed below. A preferable material for the inner layer is a 100% nylon knit jersey material. Laminated or otherwise attached to the inner layer 28 is a neoprene layer 26 comprising a backing preferably constructed of a blend of 70% styrene butadiene rubber and 30% neoprene, although other neoprenes and neoprene blends may also be used. The outer layer 24 is preferably constructed of a loop material, such as a generic version of VELCRO® that is known in the art. The combined thickness of the body 10 is preferably about 0.205 inch +/−15% at 0.5 psi, although other thicknesses are possible depending on the desired characteristics and application of the brace. In one embodiment, the material forming the body 10 has a weight of 30.0 oz/yd² +/−5%, an 85% elongation +/−15% in the cross machine direction, and a maximum shrinkage of 3%.

The body 10 includes opposing lateral ends 34, 38 and opposing top and bottom edges 32, 36, respectively. According to one embodiment, the area defined by the lateral ends and edges is approximately 10″×10 ″, although the sizing may vary depending on the application and desired wearer of the knee stabilizer 5. However, it is preferred that the body 10 as defined above remains generally square in dimension. Advantageously, the lateral end 34 is generally flat or linear and preferably forms right angles to the edges 32, 36. In one embodiment, the opposing side edges are substantially parallel to one another, although a slight taper occurs at a pair of integrated straps 11, 12, as discussed below. The body 10 also defines a patellar opening 14 that is located generally in the center of the body. In one embodiment, a patellar reinforcement ring 15 is sewn or secured to the inside surface 28 of the body for providing additional support to the patella or kneecap. In one embodiment, the reinforcement ring 15 is constructed of a distinct and separate layer of laminate material having a foam core, such as the middle layer 26 of the body 10. It is also possible that the knee stabilizer 5 may be constructed out of different materials and have more or less layers depending on many factors, including cost, use or application, and the like.

The knee stabilizer 5 also includes a pair of laterally extending top and bottom straps 11, 12, respectively, that are integral with the body 10 and extend from the lateral end 38 of the body and define a gap 40 therebetween. In one embodiment, each of the integrated straps has a hook material tab 13 attached thereto. The hook material tab 13 is also a generic version of a VELCRO® strap material and is known in the art. In one embodiment, one or more of the hook material tabs 13 have an outer surface 21 that is highly retroflective, such that an illumination is effected when struck by light in a dark ambient environment such that the light is directed back towards the source. As mentioned above, the integrated straps 11, 12 have a slightly tapering outer edge. The inner edge of edge of the straps 11, 12 is also tapering or curved such that the straps meet to define the lateral end 38 of the body 10. The gap 40 according to one embodiment is about 4 inches at the widest point, which is near the hook material tabs 13 of the straps 11, 12.

In addition, the body 10 defines one or more openings 16 positioned generally near the base of the integrated straps 11, 12 and adapted for collapsing during knee flexion to eliminate bunching or binding of the knee stabilizer 5 during activity. In one embodiment, the openings 16 are approximately 1 inch elliptical openings and positioned at approximately 45° to an imaginary lateral axis extending through the patellar opening of the body 10. Other shapes are possible for the openings 16, such as trapezoidal or circular.

The knee stabilizer 5 also includes a plurality of flexible stays 19 that are housed or encapsulated, according to one embodiment, in respective stay covers 18. It is also possible that the flexible stays are integrated into the body 10 or fastened or secured using other devices, such as additional straps, pads of hook material on the stays 19, or 30 the like. As shown, however, the flexible stays 19 are encapsulated in respective stay covers 18 that are sewn or secured to the outer surface 24 of the body 10. The stay covers 18 are constructed of a loop material, such as a lightly napped knit or unbroken loop material with a 73 cup backing, such as style 1760 produced by Gehring Tricot Corp of Dolgeville, N.Y. The stay covers 18 are preferably compatible with hook style fasteners as described herein. The stay covers 18 may have a straight or curved shape for receiving the stays, and the stay covers preferably extend along the medial and lateral sides of the patellar opening 14. In one embodiment, the stay covers 18 have a curved or angled shape toward the patellar opening 14, i.e., where the base of the curve is towards the patellar opening. Other shapes are possible for the stay covers 18 and the pockets formed by the stay covers for receiving the stays 19 depending on the desired size and shape of the stays. In one embodiment, each stay cover 18 is separated, such as by stitching, into respective pockets for receiving a pair of flexible stays.

As shown in the drawings, the flexible stays 19 are at least partially, and preferably completely, encapsulated or housed inside the stay covers 18. In this manner, the stays 19 are rendered relatively immobile on the medial and lateral sides of the body 10 and provide reinforcement for the knee. In addition, the stays 19 act as a supplement to the wearer's proprioception, allowing the wearer to sense and react to excessive bending of the knee 9. The flexible stays 19 are preferably formed of a metal or polymer, such as nylon or polyethylene. Specifically, one preferred embodiment of the flexible stays includes an overlapping metal coil 20 that is straight or somewhat arcuate or curved for allowing flexion of the knee and a normal leg position. Similarly, another preferred embodiment for the flexible stay includes an elongate polymeric member 22 having a plurality of ribs or ridges along a straight or curved or arcuate shape that allows for flexion and a curve resembling normal leg position. As shown, the flexible stays 19 and stay covers 18 have a somewhat biased shape, such as a 5°-15° bend on each end, biased toward the patellar opening 14 such that when the knee stabilizer 5 is around the wearer's knee the stays conform to a more natural position compared to flat or linear stays. However, it may also be possible for the stays 19 to be straight, but held in a bent or curved shape by the stay covers 18. Preferably, the each of the stays 19 has a thickness of about 0.10 inch, a width of about 0.38 inch, and a length of about 7.5 inches, although these dimensions can vary according to the particular size and requirements of the brace and the intended wearer.

The knee stabilizer 5 also includes a third, middle strap 17 that extends from the lateral edge 34 of the body 10 in a direction opposite the integrated straps 11, 12 and arranged to wrap behind a wearer's knee and overlap the lateral edge 38 in the gap 40 defined by the upper and lower straps. Advantageously, the middle strap 17 is not integrally formed with the body, but rather is formed of a separate material and affixed, which is defined herein as being sewn or otherwise secured to the body 10, such as by gluing or fastening, in a subsequent processing step. In one embodiment, the middle strap 17 is a 2 inch wide nylon monofilament/polyester knit elastic material, such as PN 805N or 893N, supplied by George C. Moore Co., Greensboro, N.C., and has an elongation capacity of at least 50%. Preferably, the middle strap 17 has an elongation capacity greater than that of the body 10. In addition, the middle strap 17 of the knee stabilizer 5 includes a hook material tab 13 sewn to its distal end and secured at its proximate end to the outer surface 24 of the body 10. In a preferred embodiment, the middle strap 17 is secured to the body 10 at a position spaced inwardly from the lateral edge 34, which allows for greater adjustability and control when applying the knee stabilizer 5. More specifically, the middle strap 17 is secured to the outer surface 24 of the body 10 such that the proximate end of the strap is interposed between one of the stay covers 18 and the body 10, whereby the middle strap 17 and the stay cover share the same stitching and therefore create manufacturing efficiencies.

Advantageously, these and other manufacturing efficiencies that are obtained would not be present if the middle strap were formed as part of the body 10. More specifically, the manufacture of the knee stabilizer 5 includes cutting the body 10 of the knee stabilizer from a wide and flexible sheet of laminate material, whereby multiple body pieces are arranged across the width of the sheet of laminate material and cut or punched therefrom. In conventional knee supports, the middle strap that is formed integrally with the body results in excessive overall length of the body, and therefore typically only two body pieces can be placed across the laminate sheet. By forming the middle strap 17 as a separate piece and securing it to the body 10 in a later manufacturing step, the overall dimensions of the body 10 are smaller and simpler, whereby three or more body pieces can be placed across the laminate sheet. This greatly reduces waste and improves efficiency of the manufacturing process, particularly by eliminating wasted material that would be present around an integrated middle strap. Accordingly, substantial costs savings are effected by practicing the methods of the present invention.

By way of example, an integrated middle strap having a dimension of 2×5 inches and a body having a width of 10 inches would create two generally rectangular areas of waste having dimensions of 4×5 inches, which greatly adds to the cost and complexity of manufacturing such a brace. A further advantage of the middle strap 17 being a separate piece of material is that a more advantageous material may be used having properties different than those of the body 10 and integrated straps 11, 12 that would not be available to conventional knee supports.

The knee stabilizer 5 is applied to a wearer's knee area by positioning the stabilizer such that the patella registers with the patellar opening 14, and then wrapping the straps 11, 12 about the wearer's leg such that the upper strap 11 is positioned above the patellar opening and the lower strap 12 is positioned below the patellar opening. The middle strap 17 is then wrapped behind the knee until the hook material tab 13 of the middle strap overlaps lateral edge 38 and is secured to the outer surface 24 of the body 10. In one embodiment, the stay covers 18 have a loop material outer surface similar to that of the outer surface 24 of the body 10, such that the hook material tabs 13 can be secured to the outer surface of the body 10 and/or the outer surface of the stay covers 18. In this regard, the hook material tabs 13 of the upper and lower straps 11, 12 can be adjusted to the wearer's leg size by releasably securing the hook material tabs 13 to the outer surface 24 of the body 10 and/or the outer surface of the stay covers 18. Once in place, the flexible stays 19 provide resilient bending resistance with flexion of the knee in order to support the knee area and prevent excessive movement thereof. During such flexion, the openings 16 collapse so that the body 10 and straps 11, 12 do not bunch or hinder the wearer's comfort.

Accordingly, the knee stabilizer 5 of the present invention provides advantageous performance and manufacturing features that are not present in conventional knee supports. In particular, the middle strap 17 is formed from a separate and distinct material and is affixed or attached to the body 10 of the knee stabilizer 5, while the integral top and bottom straps 11, 12 extend in the opposite direction than the middle strap 17 so that overall compressive force is balanced among the straps without unduly restricting circulation or knee flexion. In addition, medial and lateral flexible stays 19 are positioned on opposite sides of the patellar opening 14 and preferably biased in the direction of knee flexion.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. An adjustable knee stabilizer, comprising:
a body having a center portion, opposing lateral ends, and opposing top and bottom edges, the center portion defining a patellar opening;
a pair of integrated straps spaced apart and extending from one of the lateral ends of the body, the pair of integrated straps including an upper strap adapted for wrapping around a wearer's leg and being positioned above the patellar opening in use, the pair of integrated straps including a lower strap adapted for wrapping about a wearer's leg and being positioned below the patellar opening in use;
a middle strap affixed to the body and extending therefrom opposite the pair of integrated straps, the middle strap adapted for wrapping behind a wearer's knee in use and being positioned opposite the patellar opening such that the patellar opening is exposed; and
a plurality of flexible stays adjacent the patellar opening and disposed between the top and bottom edges, the flexible stays being adapted for resiliently bending with a wearer's knee.

2. An adjustable knee stabilizer according to claim 1, wherein the body further defines at least one collapsible opening proximate one of the lateral ends.

3. An adjustable knee stabilizer according to claim 1, wherein the body is a laminate comprising a neoprene layer and a loop material outer layer.

4. An adjustable knee stabilizer according to claim 3, wherein the body is a laminate comprising a nylon inner layer, a neoprene blend middle layer, and a loop material outer layer.

5. An adjustable knee stabilizer according to claim 3, further comprising respective hook material tabs attached to the upper strap, middle strap, and lower strap, the hook material tabs being adapted for releasably attaching the straps to the body to maintain tightness of the knee stabilizer in use about a wearer's leg.

6. An adjustable knee stabilizer according to claim 5, wherein the tabs have a retroflective outer surface.

7. An adjustable knee stabilizer according to claim 1, wherein the middle strap is sewn to the body.

8. An adjustable knee stabilizer according to claim 1, further comprising a pair of opposing stay covers attached to an outer surface of the body, the stay covers at least partially encapsulating the flexible stays and extending in an arc towards the patellar opening.

9. An adjustable knee stabilizer according to claim 8, wherein the opposing stay covers have a loop material outer surface.

10. An adjustable knee stabilizer according to claim 8, wherein at least one of the opposing stay covers is divided into two pockets for accommodating a pair of flexible stays.

11. An adjustable knee stabilizer according to claim 8, wherein the middle strap is affixed to the body at one end such that the end is interposed between one of the stay covers and the body.

12. An adjustable knee stabilizer according to claim 1, wherein the middle strap comprises an elastic material having an elongation capacity of at least 50%, the middle strap being formed from a material distinct from the layers forming the body.

13. An adjustable knee stabilizer according to claim 1, wherein the flexible stays comprise at least one of a group consisting of an overlapping metal coil and an elongate polymeric member.

14. An adjustable knee stabilizer according to claim 2, wherein the at least one collapsible opening includes a pair of collapsible openings having an elliptical shape and arranged such that the openings collapse in use during flexion of a user's knee.

15. An adjustable knee stabilizer according to claim 1, further comprising a patellar reinforcement ring attached to an inner surface of the body and coaxial with the patellar opening defined by the body.

16. An adjustable knee stabilizer according to claim 1, wherein the plurality of flexible stays include a pair of flexible stays on the medial side of the patellar opening and a pair of flexible stays on the lateral side of the patellar opening.

17. An adjustable knee stabilizer according to claim 16, wherein each of the flexible stays is at least 6 inches in length and is curved at each end about 5°-15°.

18. An adjustable knee stabilizer, comprising:
a laminate body having a center portion, opposing lateral ends, and opposing top and bottom edges, the center portion defining a patellar opening and a pair of collapsible openings proximate one of the lateral ends, the body comprising an inner layer, a neoprene middle layer, and a loop material outer layer;
a pair of integrated straps spaced apart and extending from one of the lateral ends of the body, the pair of integrated straps including an upper and a lower strap, each strap having a distal end with a hook material tab attached thereto, the upper strap adapted for wrapping around a wearer's leg and being positioned above the patellar opening in use, and the lower strap adapted for wrapping around a wearer's leg and being positioned below the patellar opening in use such that the hook material tabs releasably attach the upper and lower straps to the body to maintain tightness of the knee stabilizer about the wearer's leg;
a middle strap affixed to the body and extending therefrom opposite the pair of integrated straps, the middle strap having a distal end with a hook material tab attached thereto and adapted for wrapping behind a wearer's knee and being positioned opposite the patellar opening in use such that the patellar opening is exposed; and
a plurality of opposing stay covers attached to an outer surface of the body, each stay cover encapsulating at least one flexible stay that is adapted for resiliently bending with a wearer's knee, the stay covers having a loop material outer surface adapted for receiving the hook material tabs.

19. An adjustable knee stabilizer according to claim 18, wherein the middle strap is affixed to the body at one end such that the end is interposed between one of the stay covers and the body.

20. An adjustable knee stabilizer according to claim 18, wherein the tabs have a retroflective outer surface.

21. An adjustable knee stabilizer according to claim 18, wherein the at least one flexible stay is selected from a group consisting of an overlapping metal coil and an elongate polymeric member.

22. An adjustable knee stabilizer according to claim 18, wherein the pair of collapsible openings have an elliptical shape and arranged such that the openings collapse in use during flexion of a user's knee.

23. An adjustable knee stabilizer according to claim 18, further comprising a patellar reinforcement ring attached to an inner surface of the body and coaxial with the patellar opening defined by the body.

24. An adjustable knee stabilizer according to claim 18, wherein the at least one flexible stay comprises a flexible polymeric member having a plurality of ribs and a radius of curvature adapted for resiliently bending with a wearer's knee, and wherein the stay covers have a similar radius of curvature as the at least one flexible stay.

25. An adjustable knee stabilizer according to claim 18, wherein the at least one flexible stay is straight and at least one of the stay covers has a curved shape.

26. An adjustable knee stabilizer according to claim 18, wherein the at least one flexible stays includes a pair of flexible stays on the medial side of the patellar opening and a pair of flexible stays on the lateral side of the patellar opening.

27. An adjustable knee stabilizer according to claim 26, wherein at least one of the pair of flexible stays is at least 6 inches in length and is curved at each end about 5°-15°.

28. A method of manufacturing an adjustable knee stabilizer, comprising:
providing a flexible sheet of laminate material having at least a neoprene layer;
cutting a body of the knee stabilizer from the flexible sheet, the body portion defining a patellar opening and having a flat lateral end and opposing top and bottom edges, the body extending into a pair of integral straps opposite the flat lateral end that define a gap therebetween;
securing at least one flexible stay to a lateral and medial side of the patellar opening; and
affixing a middle strap to the body such that the middle strap extends from the body opposite the pair of integral straps and positioned across the body from the gap defined by the integral straps.

29. A method according to claim 28, wherein the providing step includes providing a laminate material also having an inner layer and a loop material outer layer.

30. A method according to claim 29, further comprising securing corresponding hook material tabs to each of the middle strap and integral straps that cooperate with the loop material layer of the body.

31. A method according to claim 29, wherein the securing step includes attaching a respective stay cover to the loop material layer of the body such that the stay cover at least partially encapsulates the flexible stay.

32. A method according to claim 28, wherein the affixing step includes sewing the middle strap to the body proximate the flat lateral end, the middle strap being formed from a material distinct from the layers forming the body and having an elongation capacity greater than that of the body.

33. A method according to claim 31, wherein the affixing step includes sewing the middle strap to the body and the stay cover such that the middle strap is interposed therebetween.

34. A method according to claim 30, further comprising attaching a retroflective material to at least one of the hook material tabs.

35. A method according to claim 28, wherein the cutting step includes cutting at least one opening in the body proximate the integral straps, the opening positioned and adapted for collapsing during use of the knee stabilizer about a wearer's knee.
